(19) United States
(12) Patent Application Publication
(10) Pub. No.: US 2025/0140403 A1
(43) Pub. Date: May 1, 2025

(54) TRAINED COMPUTER MODEL FOR GENERATING AGGREGATED HEALTH SCORE FOR A BUSINESS USER OF AN ONLINE SYSTEM

(71) Applicant: Maplebear Inc., San Francisco, CA (US)

(72) Inventors: Shaun Navin Maharaj, Vaughan (CA); Brent Scheibelhut, Toronto (CA); Mark Oberemk, Toronto (CA); Ryan McColeman, Toronto (CA); Ryan Martin, Toronto (CA)

(21) Appl. No.: 18/495,651

(22) Filed: Oct. 26, 2023

(51) Int. Cl. G16H 50/20; G06Q 30/0601; G06Q 40/08; G16H 10/20

(52) U.S. Cl. CPC G16H 50/20; G06Q 30/0631; G06Q 40/08; G16H 10/20

(57) ABSTRACT

A trained computer model for generating an aggregated health score for a business user of an online system. The online system obtains a set of health scores for a set of individual employees of a business user of an online system. The online system accesses a computer model of the online system trained to determine an aggregated health score for the business user. The online system applies the computer model to generate, based at least in part on the set of health scores and content of a set of orders placed by the business user, the aggregated health score for the business user. The online system causes a device of the business user to display a user interface with the aggregated health score.

### BACKGROUND

Online systems, such as online concierge systems, enable some business users (e.g., companies with user accounts) to purchase items (e.g., groceries, kitchen snacks, lunch packages, catering meals, etc.) for their employees. At the same time, some health insurance providers (e.g., through their health plans) require reporting of nutritional information about the food provided by a business user of an online concierge system to its employees. This would be a regular requirement of the health insurance provider which may alter plans for employees based on the offering of consumable items (i.e., food) by the business user. The business user may be thus responsible for purchasing groceries and other consumable items for its employees, but also for reporting on the aggregated health metrics associated with the food which is being purchased for the employees. Hence, the business user who is purchasing the food items for its employees would need a means of confirming the health impacts of the purchased food items, and how this may affect future health insurance provider plans when the time approaches for renewal. However, it is difficult to determine the aggregated health metrics for the business user at a large scale (e.g., for hundreds or thousands of employees) that varies over time. This leads to a technical problem of how to automatically generate an aggregated health score over time for a business user of an online concierge system at the large scale.

### SUMMARY

Embodiments of the present disclosure are directed to utilizing a trained computer model to automatically generate an aggregated health score for a business user of an online system (e.g., online concierge system).

In accordance with one or more aspects of the disclosure, the online system obtains a set of health scores for a set of individual employees of a business user of an online system. The online system accesses a computer model of the online system trained to determine an aggregated health score for the business user. The online system applies the computer model to generate, based at least in part on the set of health scores and content of a set of orders placed by the business user, the aggregated health score for the business user. The online system causes a device of the business user to display a user interface with the aggregated health score.

### BRIEF DESCRIPTION OF THE DRAWINGS

FIG. 1 illustrates an example system environment for an online concierge system, in accordance with one or more embodiments.

FIG. 2 illustrates an example system architecture for an online concierge system, in accordance with one or more embodiments.

FIG. 3A illustrates an example user interface with an aggregated health score for a business user of an online concierge system displayed at a user client device, in accordance with one or more embodiments.

FIG. 3B illustrates an example user interface with components of the aggregated health score displayed at the user client device, in accordance with one or more embodiments.

FIG. 4 is a flowchart for a method of using a computer model to generate an aggregated health score for a business user of an online concierge system, in accordance with one or more embodiments.

### DETAILED DESCRIPTION

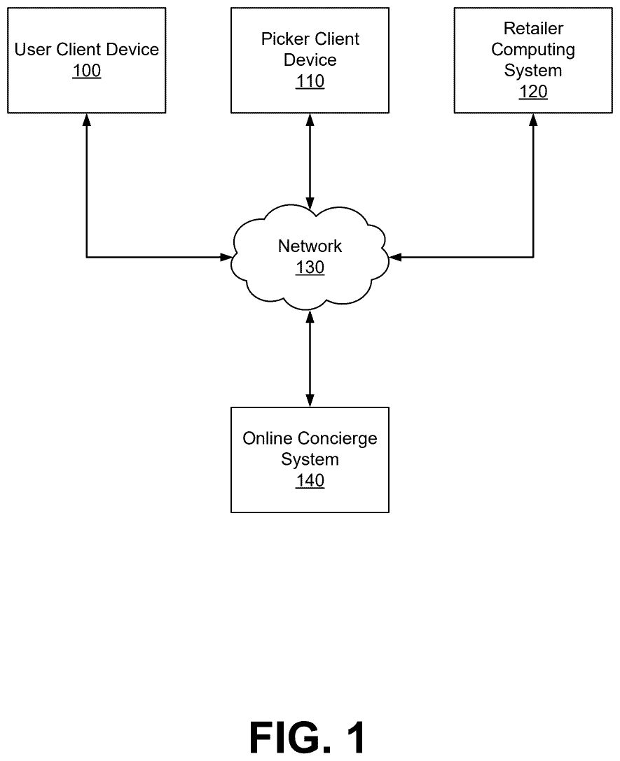

FIG. 1 illustrates an example system environment for an online concierge system 140, in accordance with one or more embodiments. The system environment illustrated in FIG. 1 includes a user client device 100, a picker client device 110, a retailer computing system 120, a network 130, and an online concierge system 140. Alternative embodiments may include more, fewer, or different components from those illustrated in FIG. 1, and the functionality of each component may be divided between the components differently from the description below. Additionally, each component may perform their respective functionalities in response to a request from a human, or automatically without human intervention.

Although one user client device 100, picker client device 110, and retailer computing system 120 are illustrated in FIG. 1, any number of users, pickers, and retailers may interact with the online concierge system 140. As such, there may be more than one user client device 100, picker client device 110, or retailer computing system 120.

The user client device 100 is a client device through which a user may interact with the picker client device 110, the retailer computing system 120, or the online concierge system 140. The user client device 100 can be a personal or mobile computing device, such as a smartphone, a tablet, a laptop computer, or desktop computer. In some embodiments, the user client device 100 executes a client application that uses an application programming interface (API) to communicate with the online concierge system 140.

A user uses the user client device 100 to place an order with the online concierge system 140. An order specifies a set of items to be delivered to the user. An “item,” as used herein, means a good or product that can be provided to the user through the online concierge system 140. The order may include item identifiers (e.g., a stock keeping unit (SKU) or a price look-up (PLU) code) for items to be delivered to the user and may include quantities of the items to be delivered. Additionally, an order may further include a delivery location to which the ordered items are to be delivered and a timeframe during which the items should be delivered. In some embodiments, the order also specifies one or more retailers from which the ordered items should be collected.

The user client device 100 presents an ordering interface to the user. The ordering interface is a user interface that the user can use to place an order with the online concierge system 140. The ordering interface may be part of a client application operating on the user client device 100. The ordering interface allows the user to search for items that are available through the online concierge system 140 and the user can select which items to add to a “shopping list.” A “shopping list,” as used herein, is a tentative set of items that the user has selected for an order but that has not yet been finalized for an order. The ordering interface allows a user to update the shopping list, e.g., by changing the quantity of items, adding or removing items, or adding instructions for items that specify how the item should be collected.

The user client device 100 may receive additional content from the online concierge system 140 to present to a user. For example, the user client device 100 may receive coupons, recipes, or item suggestions. The user client device 100 may present the received additional content to the user as the user uses the user client device 100 to place an order (e.g., as part of the ordering interface).

Additionally, the user client device 100 includes a communication interface that allows the user to communicate with a picker that is servicing the user's order. This communication interface allows the user to input a text-based message to transmit to the picker client device 110 via the network 130. The picker client device 110 receives the message from the user client device 100 and presents the message to the picker. The picker client device 110 also includes a communication interface that allows the picker to communicate with the user. The picker client device 110 transmits a message provided by the picker to the user client device 100 via the network 130. In some embodiments, messages sent between the user client device 100 and the picker client device 110 are transmitted through the online concierge system 140. In addition to text messages, the communication interfaces of the user client device 100 and the picker client device 110 may allow the user and the picker to communicate through audio or video communications, such as a phone call, a voice-over-IP call, or a video call.

The picker client device 110 is a client device through which a picker may interact with the user client device 100, the retailer computing system 120, or the online concierge system 140. The picker client device 110 can be a personal or mobile computing device, such as a smartphone, a tablet, a laptop computer, or desktop computer. In some embodiments, the picker client device 110 executes a client application that uses an application programming interface (API) to communicate with the online concierge system 140.

The picker client device 110 receives orders from the online concierge system 140 for the picker to service. A picker services an order by collecting the items listed in the order from a retailer. The picker client device 110 presents the items that are included in the user's order to the picker in a collection interface. The collection interface is a user interface that provides information to the picker on which items to collect for a user's order and the quantities of the items. In some embodiments, the collection interface provides multiple orders from multiple users for the picker to service at the same time from the same retailer location. The collection interface further presents instructions that the user may have included related to the collection of items in the order. Additionally, the collection interface may present a location of each item at the retailer, and may even specify a sequence in which the picker should collect the items for improved efficiency in collecting items. In some embodiments, the picker client device 110 transmits to the online concierge system 140 or the user client device 100 which items the picker has collected in real time as the picker collects the items.

The picker can use the picker client device 110 to keep track of the items that the picker has collected to ensure that the picker collects all of the items for an order. The picker client device 110 may include a barcode scanner that can determine an item identifier encoded in a barcode coupled to an item. The picker client device 110 compares this item identifier to items in the order that the picker is servicing, and if the item identifier corresponds to an item in the order, the picker client device 110 identifies the item as collected. In some embodiments, rather than or in addition to using a barcode scanner, the picker client device 110 captures one or more images of the item and determines the item identifier for the item based on the images. The picker client device 110 may determine the item identifier directly or by transmitting the images to the online concierge system 140. Furthermore, the picker client device 110 determines a weight for items that are priced by weight. The picker client device 110 may prompt the picker to manually input the weight of an item or may communicate with a weighing system in the retailer location to receive the weight of an item.

When the picker has collected all of the items for an order, the picker client device 110 instructs a picker on where to deliver the items for a user's order. For example, the picker client device 110 displays a delivery location from the order to the picker. The picker client device 110 also provides navigation instructions for the picker to travel from the retailer location to the delivery location. When a picker is servicing more than one order, the picker client device 110 identifies which items should be delivered to which delivery location. The picker client device 110 may provide navigation instructions from the retailer location to each of the delivery locations. The picker client device 110 may receive one or more delivery locations from the online concierge system 140 and may provide the delivery locations to the picker so that the picker can deliver the corresponding one or more orders to those locations. The picker client device 110 may also provide navigation instructions for the picker from the retailer location from which the picker collected the items to the one or more delivery locations.

In some embodiments, the picker client device 110 tracks the location of the picker as the picker delivers orders to delivery locations. The picker client device 110 collects location data and transmits the location data to the online concierge system 140. The online concierge system 140 may transmit the location data to the user client device 100 for display to the user, so that the user can keep track of when their order will be delivered. Additionally, the online concierge system 140 may generate updated navigation instructions for the picker based on the picker's location. For example, if the picker takes a wrong turn while traveling to a delivery location, the online concierge system 140 determines the picker's updated location based on location data from the picker client device 110 and generates updated navigation instructions for the picker based on the updated location.

In one or more embodiments, the picker is a single person who collects items for an order from a retailer location and delivers the order to the delivery location for the order. Alternatively, more than one person may serve the role as a picker for an order. For example, multiple people may collect the items at the retailer location for a single order. Similarly, the person who delivers an order to its delivery location may be different from the person or people who collected the items from the retailer location. In these embodiments, each person may have a picker client device 110 that they can use to interact with the online concierge system 140.

Additionally, while the description herein may primarily refer to pickers as humans, in some embodiments, some or all of the steps taken by the picker may be automated. For example, a semi- or fully-autonomous robot may collect items in a retailer location for an order and an autonomous vehicle may deliver an order to a user from a retailer location.

The retailer computing system 120 is a computing system operated by a retailer that interacts with the online concierge system 140. As used herein, a “retailer” is an entity that operates a “retailer location,” which is a store, warehouse, or other building from which a picker can collect items. The retailer computing system 120 stores and provides item data to the online concierge system 140 and may regularly update the online concierge system 140 with updated item data. For example, the retailer computing system 120 provides item data indicating which items are available at a particular retailer location and the quantities of those items. Additionally, the retailer computing system 120 may transmit updated item data to the online concierge system 140 when an item is no longer available at the retailer location. Additionally, the retailer computing system 120 may provide the online concierge system 140 with updated item prices, sales, or availabilities. Additionally, the retailer computing system 120 may receive payment information from the online concierge system 140 for orders serviced by the online concierge system 140. Alternatively, the retailer computing system 120 may provide payment to the online concierge system 140 for some portion of the overall cost of a user's order (e.g., as a commission).

The user client device 100, the picker client device 110, the retailer computing system 120, and the online concierge system 140 can communicate with each other via the network 130. The network 130 is a collection of computing devices that communicate via wired or wireless connections. The network 130 may include one or more local area networks (LANs) or one or more wide area networks (WANs). The network 130, as referred to herein, is an inclusive term that may refer to any or all of standard layers used to describe a physical or virtual network, such as the physical layer, the data link layer, the network layer, the transport layer, the session layer, the presentation layer, and the application layer. The network 130 may include physical media for communicating data from one computing device to another computing device, such as multiprotocol label switching (MPLS) lines, fiber optic cables, cellular connections (e.g., 3G, 4G, or 5G spectra), or satellites. The network 130 also may use networking protocols, such as TCP/IP, HTTP, SSH, SMS, or FTP, to transmit data between computing devices. In some embodiments, the network 130 may include Bluetooth or near-field communication (NFC) technologies or protocols for local communications between computing devices. The network 130 may transmit encrypted or unencrypted data.

The online concierge system 140 is an online system by which users can order items to be provided to them by a picker from a retailer. The online concierge system 140 receives orders from a user client device 100 through the network 130. The online concierge system 140 selects a picker to service the user's order and transmits the order to a picker client device 110 associated with the picker. The picker collects the ordered items from a retailer location and delivers the ordered items to the user. The online concierge system 140 may charge a user for the order and provide portions of the payment from the user to the picker and the retailer.

As an example, the online concierge system 140 may allow a user to order groceries from a grocery store retailer. The user's order may specify which groceries they want delivered from the grocery store and the quantities of each of the groceries. The user client device 100 transmits the user's order to the online concierge system 140 and the online concierge system 140 selects a picker to travel to the grocery store retailer location to collect the groceries ordered by the user. Once the picker has collected the groceries ordered by the user, the picker delivers the groceries to a location transmitted to the picker client device 110 by the online concierge system 140.

The online concierge system 140 maintains an account (i.e., user profile) for a business user (e.g., partner business or company with hundreds or thousands of employees) that purchases consumable items (e.g., groceries, kitchen snacks, lunch packages, catering meals, etc.) for its employees. In the same manner as an individual user of the online concierge system 140 interacts with the online concierge system 140, the business user of the online concierge system 140 utilizes a user client device to order consumable items for its employees. To understand various health-factors associated with the consumable items that are being purchased for the employees, the online concierge system 140 generates an aggregated health score for the business user by applying a computer model (e.g., machine-learning model) that is trained based on health scores computed from individual employees of the business user. The health scores of the individual employees may be obtained, e.g., from individual accounts of the employees linked to the online concierge system 140, health surveys, biometric measurements, or some combination thereof. The online concierge system 140 may report the aggregated health score over time (e.g., aggregated reports of nutritional information of purchased items) to the business user. Also, the online concierge system 140 may report (e.g., upon obtaining an agreement from the business user) the aggregated health score over time to a health insurance provider. Additionally, the online concierge system 140 may use the trends in various dimensions of the aggregated health score (e.g., a level of protein over time associated with items consumed by employees of the business user, level of sugar over time associated with the consumed items, etc.) to rank suggested items for purchase by the business user. The online concierge system 140 may also apply certain established rules to flag one or more issues to the business user at checkout time, such as when a current order violates one or more of the established rules or otherwise trigger the established rules.

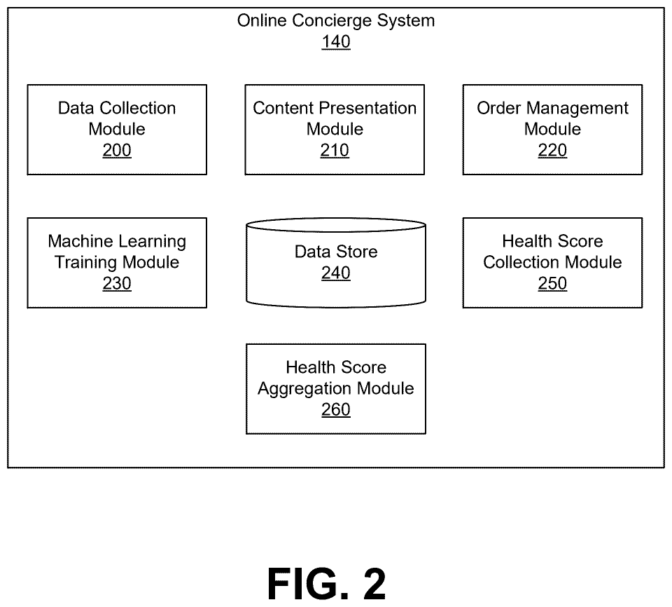

Hence, the online concierge system 140 utilizes the offerings of a grocery delivery service as the base for maintaining a health profile of the business user. The online concierge system 140 maintains a profile of the business user, and based on the business user profile, the online concierge system 140 creates an aggregated health profile of the business user and draws inferences about the overall health of employees of the business user. Based on the aggregated health profile of the business user (e.g., a set of health scores or metrics), the online concierge system 140 provides suggestions for consumable items that fall within the health metrics of the created health profile of the business user. The online concierge system 140 may generate and maintain the aggregated health profile of the business user by consistently monitoring both past orders of the business user and past orders of employees who frequently visit a work environment (e.g., office) of the business user. If the business user recognizes that some employees are often at the work environment, the online concierge system 140 may build a profile of the frequent in-office employees and build one or more specific carts for the business user, e.g., snacks for office cart, Friday food items, etc. Additionally, the online concierge system 140 may provide feedback to the business user for future orders to notify the business user with in-context suggestions. In this manner, the online concierge system 140 may ensure that the best possible health choices associated with purchased consumable items are made by the business user. The online concierge system 140 is described in further detail below with regards to FIG. 2.

FIG. 2 illustrates an example system architecture for an online concierge system 140, in accordance with some embodiments. The system architecture illustrated in FIG. 2 includes a data collection module 200, a content presentation module 210, an order management module 220, a machine-learning training module 230, a data store 240, a health score collection module 250, and a health score aggregation module 260. Alternative embodiments may include more, fewer, or different components from those illustrated in FIG. 2, and the functionality of each component may be divided between the components differently from the description below. Additionally, each component may perform their respective functionalities in response to a request from a human, or automatically without human intervention.

The data collection module 200 collects data used by the online concierge system 140 and stores the data in the data store 240. The data collection module 200 may only collect data describing a user if the user has previously explicitly consented to the online concierge system 140 collecting data describing the user. Additionally, the data collection module 200 may encrypt all data, including sensitive or personal data, describing users.

For example, the data collection module 200 collects user data, which is information or data that describe characteristics of a user. For example, the data collection module 200 may collect the user data that include a user's name, address, shopping preferences, favorite items, or stored payment instruments. The data collection module 200 may further collect the user data that include default settings established by the user, such as a default retailer/retailer location, payment instrument, delivery location, or delivery timeframe. The data collection module 200 may collect the user data from sensors on the user client device 100 or based on the user's interactions with the online concierge system 140.

The data collection module 200 also collects item data, which is information or data that identifies and describes items that are available at a retailer location. The data collection module 200 may collect the item data that include item identifiers for items that are available and may include quantities of items associated with each item identifier. Additionally, the data collection module 200 may collect the item data that also include attributes of items such as the size, color, weight, stock keeping unit (SKU), or serial number for the item. The data collection module 200 may collect the item data that further include purchasing rules associated with each item, if they exist. For example, age-restricted items such as alcohol and tobacco are flagged accordingly in the item data. The data collection module 200 may further collect the item data that also include information that is useful for predicting the availability of items in retailer locations. For example, for each item-retailer combination (a particular item at a particular warehouse), the item data may include a time that the item was last found, a time that the item was last not found (a picker looked for the item but could not find it), the rate at which the item is found, or the popularity of the item. The data collection module 200 may collect item data from a retailer computing system 120, a picker client device 110, or the user client device 100.

An item category is a set of items that are a similar type of item. Items in an item category may be considered to be equivalent to each other or that may be replacements for each other in an order. For example, different brands of sourdough bread may be different items, but these items may be in a “sourdough bread” item category. The item categories may be human-generated and human-populated with items. The item categories also may be generated automatically by the online concierge system 140 (e.g., using a clustering algorithm).

The data collection module 200 also collects picker data, which is information or data that describes characteristics of pickers. For example, the data collection module 200 may collect the picker data for a picker that include the picker's name, the picker's location, how often the picker has serviced orders for the online concierge system 140, a user rating for the picker, which retailers the picker has collected items at, or the picker's previous shopping history. Additionally, the data collection module 200 may collect the picker data that include preferences expressed by the picker, such as their preferred retailers to collect items at, how far they are willing to travel to deliver items to a user, how many items they are willing to collect at a time, timeframes within which the picker is willing to service orders, or payment information by which the picker is to be paid for servicing orders (e.g., a bank account). The data collection module 200 collects the picker data from sensors of the picker client device 110 or from the picker's interactions with the online concierge system 140.

Additionally, the data collection module 200 collects order data, which is information or data that describes characteristics of an order. For example, order data may include item data for items that are included in the order, a delivery location for the order, a user associated with the order, a retailer location from which the user wants the ordered items collected, or a timeframe within which the user wants the order delivered. Order data may further include information describing how the order was serviced, such as which picker serviced the order, when the order was delivered, or a rating that the user gave the delivery of the order. In some embodiments, the order data includes user data for users associated with the order, such as user data for a user who placed the order or picker data for a picker who serviced the order.

The content presentation module 210 selects content for presentation to a user. For example, the content presentation module 210 selects which items to present to a user while the user is placing an order. The content presentation module 210 generates and transmits an ordering interface for the user to order items. The content presentation module 210 populates the ordering interface with items that the user may select for adding to their order. In some embodiments, the content presentation module 210 presents a catalog of all items that are available to the user, which the user can browse to select items to order. The content presentation module 210 also may identify items that the user is most likely to order and present those items to the user. For example, the content presentation module 210 may score items and rank the items based on their scores. The content presentation module 210 displays the items with scores that exceed some threshold (e.g., the top n items or the p percentile of items).

The content presentation module 210 may use an item selection model to score items for presentation to a user. An item selection model is a machine-learning model that is trained to score items for a user based on item data for the items and user data for the user. For example, the item selection model may be trained to determine a likelihood that the user will order the item. In some embodiments, the item selection model uses item embeddings describing items and user embeddings describing users to score items. These item embeddings and user embeddings may be generated by separate machine-learning models and may be stored in the data store 240.

In some embodiments, the content presentation module 210 scores items based on a search query received from the user client device 100. A search query is free text for a word or set of words that indicate items of interest to the user. The content presentation module 210 scores items based on a relatedness of the items to the search query. For example, the content presentation module 210 may apply natural language processing (NLP) techniques to the text in the search query to generate a search query representation (e.g., an embedding) that represents characteristics of the search query. The content presentation module 210 may use the search query representation to score candidate items for presentation to a user (e.g., by comparing a search query embedding to an item embedding).

In some embodiments, the content presentation module 210 scores items based on a predicted availability of an item. The content presentation module 210 may use an availability model to predict the availability of an item. An availability model is a machine-learning model that is trained to predict the availability of an item at a particular retailer location. For example, the availability model may be trained to predict a likelihood that an item is available at a retailer location or may predict an estimated number of items that are available at a retailer location. The content presentation module 210 may apply a weight to the score for an item based on the predicted availability of the item. Alternatively, the content presentation module 210 may filter out items from presentation to a user based on whether the predicted availability of the item exceeds a threshold.

The order management module 220 manages orders for items from users. The order management module 220 receives orders from a user client device 100 and assigns the orders to pickers for service based on picker data. For example, the order management module 220 assigns an order to a picker based on the picker's location and the location of the retailer from which the ordered items are to be collected. The order management module 220 may also assign an order to a picker based on how many items are in the order, a vehicle operated by the picker, the delivery location, the picker's preferences on how far to travel to deliver an order, the picker's ratings by users, or how often a picker agrees to service an order.

In some embodiments, the order management module 220 determines when to assign an order to a picker based on a delivery timeframe requested by the user with the order. The order management module 220 computes an estimated amount of time that it would take for a picker to collect the items for an order and deliver the ordered items to the delivery location for the order. The order management module 220 assigns the order to a picker at a time such that, if the picker immediately services the order, the picker is likely to deliver the order at a time within the requested timeframe. Thus, when the order management module 220 receives an order, the order management module 220 may delay in assigning the order to a picker if the requested timeframe is far enough in the future (i.e., the picker may be assigned at a later time and is still predicted to meet the requested timeframe).

When the order management module 220 assigns an order to a picker, the order management module 220 transmits the order to the picker client device 110 associated with the picker. The order management module 220 may also transmit navigation instructions from the picker's current location to the retailer location associated with the order. If the order includes items to collect from multiple retailer locations, the order management module 220 identifies the retailer locations to the picker and may also specify a sequence in which the picker should visit the retailer locations.

The order management module 220 may track the location of the picker through the picker client device 110 to determine when the picker arrives at the retailer location. When the picker arrives at the retailer location, the order management module 220 transmits the order to the picker client device 110 for display to the picker. As the picker uses the picker client device 110 to collect items at the retailer location, the order management module 220 receives item identifiers for items that the picker has collected for the order. In some embodiments, the order management module 220 receives images of items from the picker client device 110 and applies computer-vision techniques to the images to identify the items depicted by the images. The order management module 220 may track the progress of the picker as the picker collects items for an order and may transmit progress updates to the user client device 100 that describe which items have been collected for the user's order.

In some embodiments, the order management module 220 tracks the location of the picker within the retailer location. The order management module 220 uses sensor data from the picker client device 110 or from sensors in the retailer location to determine the location of the picker in the retailer location. The order management module 220 may transmit, to the picker client device 110, instructions to display a map of the retailer location indicating where in the retailer location the picker is located. Additionally, the order management module 220 may instruct the picker client device 110 to display the locations of items for the picker to collect, and may further display navigation instructions for how the picker can travel from their current location to the location of a next item to collect for an order.

The order management module 220 determines when the picker has collected all of the items for an order. For example, the order management module 220 may receive a message from the picker client device 110 indicating that all of the items for an order have been collected. Alternatively, the order management module 220 may receive item identifiers for items collected by the picker and determine when all of the items in an order have been collected. When the order management module 220 determines that the picker has completed an order, the order management module 220 transmits the delivery location for the order to the picker client device 110. The order management module 220 may also transmit navigation instructions to the picker client device 110 that specify how to travel from the retailer location to the delivery location, or to a subsequent retailer location for further item collection. The order management module 220 tracks the location of the picker as the picker travels to the delivery location for an order, and updates the user with the location of the picker so that the user can track the progress of the order. In some embodiments, the order management module 220 computes an estimated time of arrival of the picker at the delivery location and provides the estimated time of arrival to the user.

In some embodiments, the order management module 220 facilitates communication between the user client device 100 and the picker client device 110. As noted above, a user may use a user client device 100 to send a message to the picker client device 110. The order management module 220 receives the message from the user client device 100 and transmits the message to the picker client device 110 for presentation to the picker. The picker may use the picker client device 110 to send a message to the user client device 100 in a similar manner.

The order management module 220 coordinates payment by the user for the order. The order management module 220 uses payment information provided by the user (e.g., a credit card number or a bank account) to receive payment for the order. In some embodiments, the order management module 220 stores the payment information for use in subsequent orders by the user. The order management module 220 computes a total cost for the order and charges the user that cost. The order management module 220 may provide a portion of the total cost to the picker for servicing the order, and another portion of the total cost to the retailer.

The machine-learning training module 230 trains machine-learning models used by the online concierge system 140. The online concierge system 140 may use machine-learning models to perform functionalities described herein. Example machine-learning models include regression models, support vector machines, naïve bayes, decision trees, k nearest neighbors, random forest, boosting algorithms, k-means, and hierarchical clustering. The machine-learning models may also include neural networks, such as perceptrons, multilayer perceptrons, convolutional neural networks, recurrent neural networks, sequence-to-sequence models, generative adversarial networks, or transformers. A machine-learning model may include components relating to these different general categories of model, which may be sequenced, layered, or otherwise combined in various configurations. While the term “machine-learning model” may be broadly used herein to refer to any kind of machine-learning model, the term is generally limited to those types of models that are suitable for performing the described functionality. For example, certain types of machine-learning models can perform a particular functionality based on the intended inputs to, and outputs from, the model, the capabilities of the system on which the machine-learning model will operate, or the type and availability of training data for the model.

Each machine-learning model includes a set of parameters. The set of parameters for a machine-learning model are parameters that the machine-learning model uses to process an input to generate an output. For example, a set of parameters for a linear regression model may include weights that are applied to each input variable in the linear combination that comprises the linear regression model. Similarly, the set of parameters for a neural network may include weights and biases that are applied at each neuron in the neural network. The machine-learning training module 230 generates the set of parameters (e.g., the particular values of the parameters) for a machine-learning model by “training” the machine-learning model. Once trained, the machine-learning model uses the set of parameters to transform inputs into outputs.

The machine-learning training module 230 trains a machine-learning model based on a set of training examples. Each training example includes input data to which the machine-learning model is applied to generate an output. For example, each training example may include user data, picker data, item data, or order data. In some cases, the training examples also include a label which represents an expected output of the machine-learning model. In these cases, the machine-learning model is trained by comparing its output from input data of a training example to the label for the training example. In general, during training with labeled data, the set of parameters of the model may be set or adjusted to reduce a difference between the output for the training example (given the current parameters of the model) and the label for the training example.

The machine-learning training module 230 may apply an iterative process to train a machine-learning model whereby the machine-learning training module 230 updates parameter values of the machine-learning model based on each of the set of training examples. The training examples may be processed together, individually, or in batches. To train a machine-learning model based on a training example, the machine-learning training module 230 applies the machine-learning model to the input data in the training example to generate an output based on a current set of parameter values. The machine-learning training module 230 scores the output from the machine-learning model using a loss function. A loss function is a function that generates a score for the output of the machine-learning model such that the score is higher when the machine-learning model performs poorly and lower when the machine-learning model performs well. In cases where the training example includes a label, the loss function is also based on the label for the training example. Some example loss functions include the mean square error function, the mean absolute error, hinge loss function, and the cross entropy loss function. The machine-learning training module 230 updates the set of parameters for the machine-learning model based on the score generated by the loss function. For example, the machine-learning training module 230 may apply gradient descent to update the set of parameters.

In one or more embodiments, the machine-learning training module 230 may retrain the machine-learning model based on the actual performance of the model after the online concierge system 140 has deployed the model to provide service to users. For example, if the machine-learning model is used to predict a likelihood of an outcome of an event, the online concierge system 140 may log the prediction and an observation of the actual outcome of the event. Alternatively, if the machine-learning model is used to classify an object, the online concierge system 140 may log the classification as well as a label indicating a correct classification of the object (e.g., following a human labeler or other inferred indication of the correct classification). After sufficient additional training data has been acquired, the machine-learning training module 230 retrains the machine-learning model using the additional training data, using any of the methods described above. This deployment and re-training process may be repeated over the lifetime use for the machine-learning model. This way, the machine-learning model continues to improve its output and adapts to changes in the system environment, thereby improving the functionality of the online concierge system 140 as a whole in its performance of the tasks described herein.

The data store 240 stores data used by the online concierge system 140. For example, the data store 240 stores user data, item data, order data, and picker data for use by the online concierge system 140. The data store 240 also stores trained machine-learning models trained by the machine-learning training module 230. For example, the data store 240 may store the set of parameters for a trained machine-learning model on one or more non-transitory, computer-readable media. The data store 240 uses computer-readable media to store data, and may use databases to organize the stored data.

The health score collection module 250 may obtain a set of health scores for a set of individual employees of a business user of the online concierge system 140. The health score collection module 250 may determine the set of health scores by scoring the set of individual employees using one or more rules for “healthy diets” that were set based on information about diets of the individual employees. The one or more rules may be obtained from, e.g., a health insurance provider or the business user. Alternatively, the health score collection module 250 may itself generate the one or more rules. The health score collection module 250 may use information about content of orders placed by the individual employees at the online concierge system 140 as a proxy for their diets. Alternatively or additionally, the health score collection module 250 may obtain health-related data directly from the individual employees, such as through surveys and/or biometric measurements associated with the individual employees (e.g., by measuring blood pressure, weight, cholesterol, etc.). Alternatively or additionally, the health score collection module 250 may sample the individual employees based on information about their attendance at a work environment (e.g., office attendance) and weight more a subset of the individual employees that are more often present at the work environment.

The health score aggregation module 260 applies a computer model (e.g., machine-learning model) trained to determine an aggregated health score for the business user. The computer model deployed by the health score aggregation module 260 may run a machine-learning algorithm to determine, based at least in part on the set of health scores for the set of individual employees obtained from the health score collection module 250 and information about a series of previous orders placed by the business user, the aggregated health score for the business user. The computer model may calculate the aggregated health score by inferring the nutritional factors across the items consumed at the work environment of the business user, and then by comparing the inferred nutritional factors against a baseline diet that is considered “healthy” for the population of employees of the business user. The computer model may determine the baseline diet by incorporating data about demographics of the employees (e.g., age, sex, health conditions, diet, etc.) to determine what is (or what is not) a healthy element of diet for the population of employees. A set of parameters for the computer model deployed by the health score aggregation module 260 may be stored on one or more non-transitory computer-readable media of the health score aggregation module 260. Alternatively, the set of parameters for the computer model deployed by the health score aggregation module 260 may be stored on one or more non-transitory computer-readable media of the data store 240.

The health score aggregation module 260 may provide inputs to the computer model, such as the health scores for the individual employees from the health score collection module 250 and the information about the series of previous orders (e.g., as available at the data store 240). The information about the one or more previous orders may include diet profile of the individual employees at the work environment of the business user (e.g., at the company's office(s)), the history of previously purchased consumable items across the business user (e.g., company) including references to products within past purchased orders, information about consumable items frequently purchased by the business user, etc.

The computer model deployed by the health score aggregation module 260 may output the aggregated health score for the business user, based on the inputs provided to the computer model. The aggregated health score determined by the computer model may represent the nutritional profile of the business user as a whole, consisting of an aggregation of a nutritional profile calculated for each employee of the business user. The aggregated health score generated by the computer model may represent the ground truth label for the aggregated health profile of the business user. Additionally, the computer model may generate an aggregated health score for each distinct work environment (e.g., distinct office) of the business user, based on health scores and diet information of individual employees at that distinct work environment. The computer model may generate the aggregated health score over time for the employee population of the business user.

In an absence of health-related data of individual employees, the computer model may assign a default value to the aggregated health score until enough health-related data of individual employees has been captured to calculate a “true” aggregated health score. In one or more embodiments, the computer model utilizes seasonality attributes of the consumable items to infer consumption of the seasonal items outside the work environment of the business user. The computer model may then use information about the inferred consumption of the seasonal items outside the work environment when generating the aggregated health score for the business user. In one or more embodiments, the computer model generates a multidimensional aggregated health score that includes multiple components, e.g., a sugar component, protein component, cholesterol component, sodium component, etc.

In one or more embodiments, the online concierge system 140 allows the individual employees of the business user to request consumable items to the business user, e.g., using their individual accounts at the online concierge system 140 and individual user client devices 100 in communication with the order management module 220. The computer model may be then populated with order history from the business user, as well as with consumable items requested by the individual employees. The health score aggregation module 260 may feed the information about consumable items requested via the individual accounts to the computer model so that the computer model may infer which individual employees are most likely to request various consumable items. This information about individual employees may be fed into “a demographics input” of the computer model, which allows for either direct input or inference of dietary restrictions, e.g., inference of vegan employees, employees seeking out gluten-free diet, etc. The information about consumable items requested via the individual accounts can be also used by the computer model to rank consumable items of a current order placed by the business user, which would be provided as a suggestion to the business user (e.g., via the user interface of the user client device 100 of the business user) to improve the current order.

If the business user is insured by a health insurance provider, the health insurance provider would typically like to know if the business user is purchasing healthy consumable items. In one or more embodiments, the online concierge system 140 is integrated with one or more health insurance providers so that the online concierge system 140 submits, to the one or more health insurance providers, information related to the aggregated health score as determined over time by the computer model. The submitted information may be exported and available to the one or more health insurance providers by means of both raw nutritional data aggregated across the employee population of the business user, as well as an aggregate rating (“trusted health score”), which the one or more health insurance providers may use to assess the health-related trends of the business user at a high level.

Upon receiving an approval from the business user, the online concierge system 140 may provide access of a business user profile to the health insurance provider, as well as a translated list of consumable items with a frequency of purchase for each consumable item. The online concierge system 140 may also save the translated list of consumable items (e.g., at the data store 240) for future retraining of the computer model (e.g., via the machine-learning training module 230). The health insurance provider may then draw inferences of consumable items of varying nutritional values and provide suggestions for consumable items (e.g., snacks/food) that fall within the health scores (e.g., health metrics) of the business user profile. The health metrics may consist of nutritional factors for the consumable items, such as: sugar, fats (saturated fats, trans fats, etc.), sodium, protein, vitamins, calories, cholesterol, etc. A separate metric for dietary restrictions may be applied after the health metrics are updated, which would filter out consumable items that are in violation of the dietary restrictions of the individual employees of the business user. The health insurance provider may update “the trusted health score” of the business user based on the business user profile and a returned health score from the health insurance provider. The health insurance provider may also assign the individual health scores to individual employees of the business user and frequently update the individual health scores based on recurrence of the individual employees to the work environment (e.g., office). The health insurance provider may maintain links to all individual employees of the business user. As aforementioned, accounts of the individual employees at the online concierge system 140 may be linked to the account of the business user at the online concierge system 140, and any profile data for an individual employee may be provided to the business user for business usage. The health insurance provider may then update health insurance rates (e.g., premiums) per individual employee of the business user based on the health scores provided for each individual employee of the business user.

The machine-learning training module 230 may train the computer model deployed by the health score aggregation module 260 to predict an aggregated health score for the business user based on order history. In one or more embodiments, the machine-learning training module 230 receives training data (e.g., from the order management module 220) that includes content of one or more previous orders assuming that the ordered items were totally ingested. In one or more other embodiments, the order management module 220 infers that some of ordered items were not ingested based on, e.g., information about reordered items. In such cases, training data for the machine-learning training module 230 may not include information about the items that were inferred as not being ingested. In one or more other embodiments, the machine-learning training module 230 may directly receive explicit data from the business user with information about which ordered items were not ingested. In such cases, the machine-learning training module 230 may not use information about the items that were not ingested for training and retraining of the computer model.

The content presentation module 210 may cause the user client device 100 of the business user to display a user interface with the aggregated health score. Furthermore, as the aggregated health score may have no absolute meaning, the content presentation module 210 may cause the user client device 100 of the business user to display a user interface a trend of the aggregated health score, i.e., the aggregated health score over time. The online concierge system 140 may report (e.g., via the network 130) the same information about the trend of the aggregated health score to the health insurance provider so that the health insurance provider can adjust premiums for the business user based on the trend of the aggregated health score. Based on the reported trend of the aggregated health score, the health insurance provider may offer, e.g., reduced premiums for business users that have aggregated health scores below a threshold score (i.e., “good health scores”), reimbursement for consumable items that significantly impact an aggregated health score, etc.

Based on the trendline of the aggregated health score, the online concierge system 140 may provide (e.g., via the order management module 220) recommendations to the business user during the purchase flow (e.g., at checkout) to move the aggregated health score for the business user in various ways. For example, if the trendline of the aggregated health score is moving in the wrong direction (e.g., decreases over time), then the online concierge system 140 may weight (e.g., via the order management module 220) the healthy options more in the purchase flow. If the aggregated health score is multidimensional, then the content presentation module 210 may cause the user client device 100 to display the user interface with a list of healthy items for that particular dimension the aggregated health score (e.g., if protein content is decreasing, then recommend more protein-based items). The content presentation module 210 may cause the user client device 100 to display the user interface with feedback for the office management staff that include summary messages in the business user's profile homepage by means of a banner, e.g., “Over the past month, the sodium content of your purchases has been higher than usual”. Additionally or alternatively, the content presentation module 210 may cause the user client device 100 to display the user interface with in-line alerts on the checkout page warning the business user, e.g., “You have been ordering higher levels of sugar in the past week”. This feedback can then be accompanied by alternatives suggested by the online concierge system 140, along with the projected outcome (reduced sugar intake, reduced sodium intake, higher protein content, etc.) of a given purchase. These alternatives could additionally be sold as sponsored placements spots, provided these sponsored products provide similar nutritional effects to the original replacements.

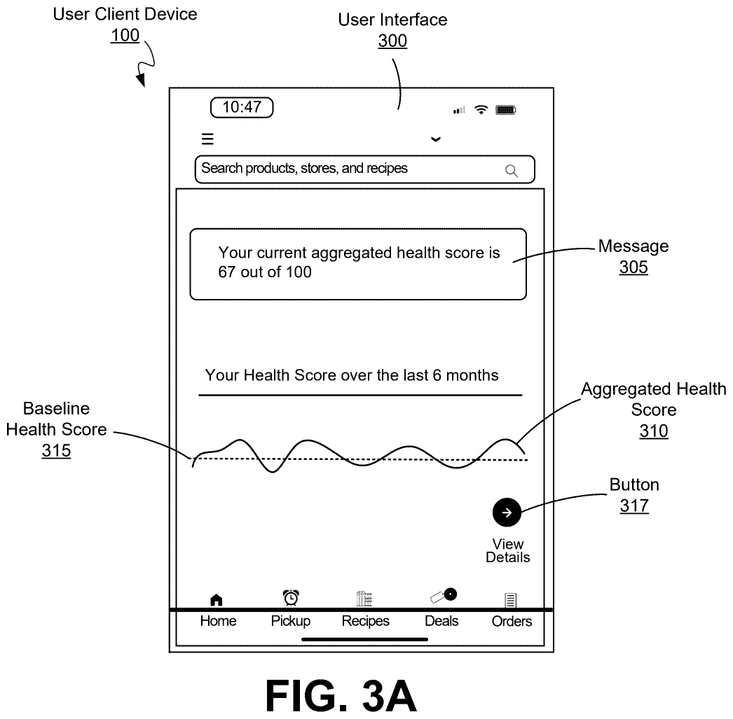

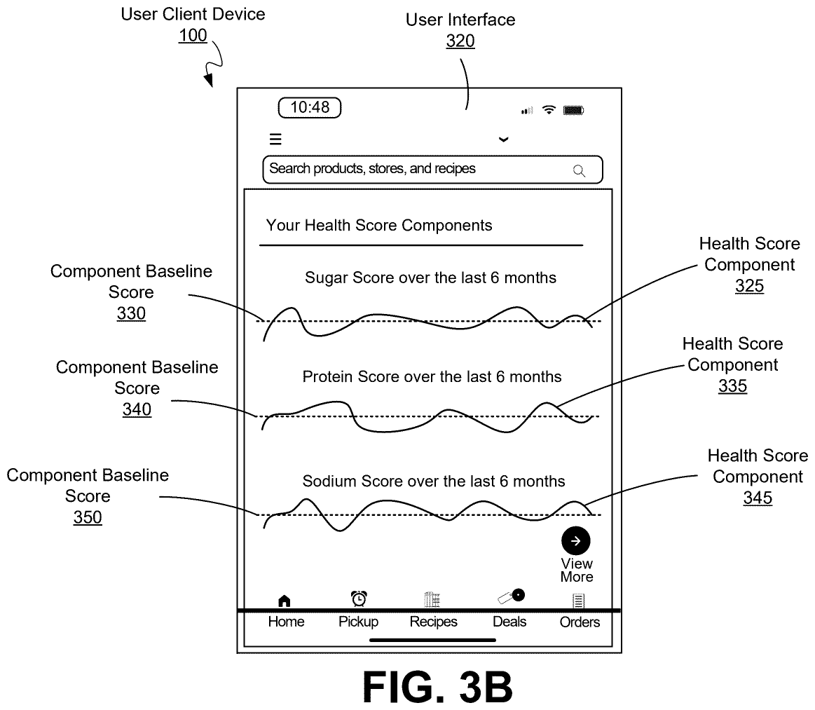

FIG. 3A illustrates an example user interface 300 with an aggregated health score for a business user of the online concierge system 140 displayed at the user client device 100, in accordance with one or more embodiments. The user interface 300 may be displayed at a home page of a business user profile at the online concierge system 140. The content presentation module 210 may cause the user client device 100 to display the user interface 300 with a message 305 that informs the business user about a current aggregated health score as determined by the computer model deployed by the health score aggregation module 260. Furthermore, the content presentation module 210 may cause the user client device 100 to display the user interface 300 further with a trendline for an aggregated health score 310 as determined by the computer model over a defined time period (e.g., over the last 6 months). The trendline for the aggregated health score 310 is overlayed on a baseline health score 315 representing a threshold for a “good” aggregated health score. Hence, the aggregated health score 310 is trending in the “right direction” when increasing and becoming higher than the baseline health score 315. In contrast, the aggregated health score 310 is trending in the “wrong direction” when decreasing and becoming less than the baseline health score 315. Information about the trendline for the aggregated health score 310 overlayed on the baseline health score 315 may be also provided (e.g., via the content presentation module 210) to a health insurance provider. The business user can view more details about the trendline of the aggregated health score 310 when selecting a button 317. Additional details about the trendline of the aggregated health score 310 are displayed at a user interface 320 of FIG. 3B.

FIG. 3B illustrates an example user interface 320 with components of the aggregated health score 310 displayed at the user client device 100, in accordance with one or more embodiments. The content presentation module 210 may cause the user client device 100 to display the user interface 320 with trendlines for health score components 325, 335, 345 of the aggregated health score 310 as determined by the computer model deployed by the health score aggregation module 260 over the defined time period (e.g., over the last 6 months). Each trendline for the health score component 325, 335, 345 is overlayed on a corresponding baseline score 330, 340, 350 representing a threshold for a “good” health score component. For example, the health score component 325 represents a health score for “sugar related intake” over the defined time period, the health score component 335 represents a health score for “protein related intake” over the defined time period, and the health score component 345 represents a health score for “sodium related intake” over the defined time period, as determined by the computer model. Hence, the health score component 325 is trending in the “right direction” when decreasing and becoming less than the component baseline score 330, and the health score component 325 is trending in the “wrong direction” when increasing and becoming higher than the component baseline score 330. The health score component 335 is trending in the “right direction” when increasing and becoming higher than the component baseline score 340, and the health score component 335 is trending in the “wrong direction” when decreasing and becoming less than the component baseline score 340. The health score component 345 is trending in the “right direction” when decreasing and becoming less than the component baseline score 350, and the health score component 345 is trending in the “wrong direction” when increasing and becoming higher than the component baseline score 350. Information about the trendlines for the health score components 325, 335, 345 overlayed on the component baseline scores 330, 340, 350 may be also provided (e.g., via the content presentation module 210) to a health insurance provider.

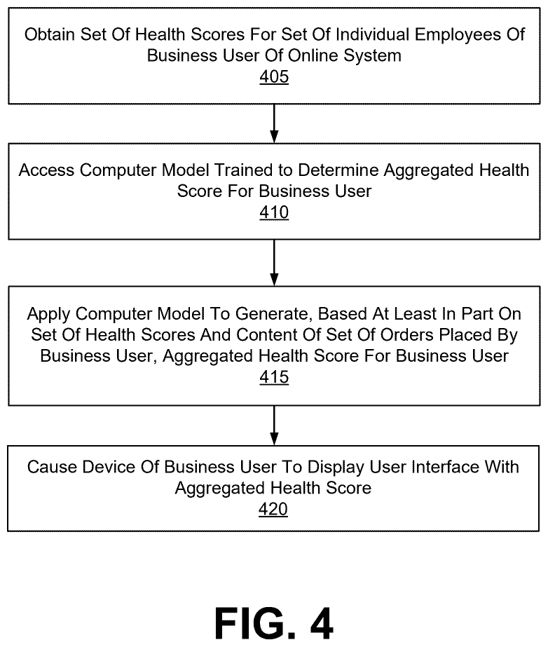

FIG. 4 is a flowchart for a method of using a computer model to generate an aggregated health score for a business user of an online concierge system, in accordance with one or more embodiments. Alternative embodiments may include more, fewer, or different steps from those illustrated in FIG. 4, and the steps may be performed in a different order from that illustrated in FIG. 4. These steps may be performed by an online concierge system (e.g., the online concierge system 140). Additionally, each of these steps may be performed automatically by the online concierge system without human intervention.

The online concierge system 140 obtains 405 (e.g., via the health score collection module 250) a set of health scores for a set of individual employees of a business user of the online concierge system 140. The online concierge system 140 may retrieve (e.g., via the health score collection module 250) health-related information from an individual account at the online concierge system 140 of each individual employee of the set of individual employees. The online concierge system 140 may generate (e.g., via the health score collection module 250), based at least in part on the retrieved health-related information, a respective health score for each individual employee of the set of individual employees. The online concierge system 140 may obtain the set of health scores by obtaining, based at least in part on a health-related survey of each individual employee of the set of individual employees, a respective health score for each individual employee of the set of individual employees. Alternatively or additionally, the online concierge system 140 may obtain the set of health scores by obtaining, based at least in part on one or more biometric measurements associated with each individual employee of the set of individual employees, a respective health score for each individual employee of the set of individual employees.

The online concierge system 140 accesses 410 (e.g., via the health score aggregation module 260) a computer model of the online concierge system 140 trained to determine an aggregated health score for the business user. The online concierge system 140 applies 415 the computer model (e.g., via the health score aggregation module 260) to generate, based at least in part on the set of health scores and content of a set of orders placed by the business user, the aggregated health score for the business user. The online concierge system 140 may apply the computer model (e.g., via the health score aggregation module 260) to generate, based on the set of health scores for the set of individual employees of the business user, a plurality of components of the aggregated health score for the business user. The online concierge system 140 may apply the computer model (e.g., via the health score aggregation module 260) to rank a list of items for purchase by the business user, based at least in part on one or more trends over time associated with one or more of the plurality of components of the aggregated health score. The online concierge system 140 may apply the computer model (e.g., via the health score aggregation module 260) to infer, based at least in part on a seasonality attribute of one or more items not being ordered by the business user, information about consumption of the one or more items by the set of individual employees. The online concierge system 140 may apply the computer model (e.g., via the health score aggregation module 260) to generate, further based on the inferred information about consumption of the one or more items, the aggregated health score for the business user.

The online concierge system 140 causes 420 (e.g., via the content presentation module 210) a device of the business user (e.g., the user client device 100) to display a user interface with the aggregated health score. The online concierge system 140 may cause (e.g., via the content presentation module 210) the device of the business user to display the user interface with the ranked list of items for inclusion into a cart of the business user. The online concierge system 140 may communicate, to a health insurance entity (e.g., health insurance provider) over a network (e.g., the network 130), information about changes of the aggregated health score over time.

The online concierge system 140 may obtain (e.g., from one or more databases of the data store 240) information about content of one or more previous orders placed by the business user. The online concierge system 140 may apply the computer model (e.g., via the health score aggregation module 260) to generate, based at least in part on the information about the content of the one or more previous orders, a training set of health scores for the set of individual employees of the business user. The online concierge system 140 may train and retrain (e.g., via the machine-learning training module 230) the computer model, based at least in part on the training set of health scores. The online concierge system 140 may obtain (e.g., from one or more databases of the data store 240) information about a portion of content of one or more previous orders placed by the business user that was not ingested. The online concierge system 140 may apply the computer model (e.g., via the health score aggregation module 260) to generate, based at least in part on the information about the portion of content that was not ingested, a training set of health scores for the set of individual employees of the business user. The online concierge system 140 may train and retrain (e.g., via the machine-learning training module 230) the computer model, based at least in part on the training set of health scores.

Embodiments of the present disclosure are directed to the online concierge system 140 that employs a computer model trained to automatically generate an aggregated health score for a business user of the online concierge system 140. The online concierge system 140 may report trends of the aggregated health score to the business user as well as to a health insurance provider. Additionally, the aggregated health score may be also utilized for recommending “healthy” consumable items to the business user.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor comprises one or more processors or processing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or.” For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A method, performed at a computer system comprising a processor and a computer-readable medium, comprising:
obtaining a set of health scores for a set of individual employees of a business user of an online system;
accessing a computer model of the online system trained to determine an aggregated health score for the business user;
applying the computer model to generate, based at least in part on the set of health scores and content of a set of orders placed by the business user, the aggregated health score for the business user; and
causing a device of the business user to display a user interface with the aggregated health score.

2. The method of claim 1, wherein obtaining the set of health scores comprises:
retrieving health-related information from an individual account at the online system of each individual employee of the set of individual employees; and
generating, based at least in part on the retrieved health-related information, a respective health score for each individual employee of the set of individual employees.

3. The method of claim 1, wherein obtaining the set of health scores comprises:
obtaining, based at least in part on a health-related survey of each individual employee of the set of individual employees, a respective health score for each individual employee of the set of individual employees.

4. The method of claim 1, wherein obtaining the set of health scores comprises:
obtaining, based at least in part on one or more biometric measurements associated with each individual employee of the set of individual employees, a respective health score for each individual employee of the set of individual employees.

5. The method of claim 1, further comprising:
obtaining information about content of one or more previous orders placed by the business user;
applying the computer model to generate, based at least in part on the information about the content of the one or more previous orders, a training set of health scores for the set of individual employees of the business user; and
training the computer model, based at least in part on the training set of health scores.

6. The method of claim 1, further comprising:
obtaining information about a portion of content of one or more previous orders placed by the business user that was not ingested;
applying the computer model to generate, based at least in part on the information about the portion of content that was not ingested, a training set of health scores for the set of individual employees of the business user; and
training the computer model, based at least in part on the training set of health scores.

7. The method of claim 1, wherein applying the computer model comprises:
applying the computer model to generate, based on the set of health scores for the set of individual employees of the business user, a plurality of components of the aggregated health score for the business user.

8. The method of claim 7, wherein applying the computer model further comprises:
ranking a list of items for purchase by the business user, based at least in part on one or more trends over time associated with one or more of the plurality of components of the aggregated health score.

9. The method of claim 8, wherein displaying the user interface further comprises:
causing the device of the business user to display the user interface with the ranked list of items for inclusion into a cart of the business user.

10. The method of claim 1, wherein applying the computer model comprises:
inferring, based at least in part on a seasonality attribute of one or more items not being ordered by the business user, information about consumption of the one or more items by the set of individual employees; and
generating, further based on the inferred information about consumption of the one or more items, the aggregated health score for the business user.

11. The method of claim 1, further comprising:
communicating, to a health insurance entity over a network, information about changes of the aggregated health score over time.

12. A computer program product comprising a non-transitory computer readable storage medium having instructions encoded thereon that, when executed by a processor, cause the processor to perform steps comprising:
obtaining a set of health scores for a set of individual employees of a business user of an online system;
accessing a computer model of the online system trained to determine an aggregated health score for the business user;
applying the computer model to generate, based at least in part on the set of health scores and content of a set of orders placed by the business user, the aggregated health score for the business user; and
causing a device of the business user to display a user interface with the aggregated health score.

13. The computer program product of claim 12, wherein the instructions further cause the processor to perform steps comprising:
retrieving health-related information from an individual account at the online system of each individual employee of the set of individual employees; and
generating, based at least in part on the retrieved health-related information, a respective health score for each individual employee of the set of individual employees.

14. The computer program product of claim 12, wherein the instructions further cause the processor to perform steps comprising:
obtaining the set of health scores by obtaining, based at least in part on a health-related survey of each individual employee of the set of individual employees, a respective health score for each individual employee of the set of individual employees.

15. The computer program product of claim 12, wherein the instructions further cause the processor to perform steps comprising:
obtaining information about content of one or more previous orders placed by the business user;
applying the computer model to generate, based at least in part on the information about the content of the one or more previous orders, a training set of health scores for the set of individual employees of the business user; and
training the computer model, based at least in part on the training set of health scores.

16. The computer program product of claim 12, wherein the instructions further cause the processor to perform steps comprising:
obtaining information about a portion of content of one or more previous orders placed by the business user that was not ingested;
applying the computer model to generate, based at least in part on the information about the portion of content that was not ingested, a training set of health scores for the set of individual employees of the business user; and
training the computer model, based at least in part on the training set of health scores.

17. The computer program product of claim 12, wherein the instructions further cause the processor to perform steps comprising:
applying the computer model to generate, based on the set of health scores for the set of individual employees of the business user, a plurality of components of the aggregated health score for the business user;
ranking a list of items for purchase by the business user, based at least in part on one or more trends over time associated with one or more of the plurality of components of the aggregated health score; and
causing the device of the business user to display the user interface with the ranked list of items for inclusion into a cart of the business user.

18. The computer program product of claim 12, wherein the instructions further cause the processor to perform steps comprising:
applying the computer model to infer, based at least in part on a seasonality attribute of one or more items not being ordered by the business user, information about consumption of the one or more items by the set of individual employees; and
applying the computer model to generate, further based on the inferred information about consumption of the one or more items, the aggregated health score for the business user.

19. The computer program product of claim 12, wherein the instructions further cause the processor to perform steps comprising:
communicating, to a health insurance entity over a network, information about changes of the aggregated health score over time.

20. A computer system comprising:
a processor; and
a non-transitory computer-readable storage medium having instructions that, when executed by the processor, cause the computer system to perform steps comprising:
obtaining a set of health scores for a set of individual employees of a business user of an online system;
accessing a computer model of the online system trained to determine an aggregated health score for the business user;
applying the computer model to generate, based at least in part on the set of health scores and content of a set of orders placed by the business user, the aggregated health score for the business user; and
causing a device of the business user to display a user interface with the aggregated health score
